(19) United States
(12) Patent Application Publication
(10) Pub. No.: US 2005/0032841 A1
(43) Pub. Date: Feb. 10, 2005

(54) Therapeutic compositions and methods of treating glycolipid storage related disorders

(76) Inventor: Steven Walkley, South Salem, NY (US)

Correspondence Address: KLAUBER & JACKSON, 411 HACKENSACK AVENUE, HACKENSACK, NJ, 07601, US

(21) Appl. No.: 10/619,378

(22) Filed: Jul. 14, 2003

Related U.S. Application Data
(63) Continuation-in-part of application No. 10/042,527, filed on Oct. 19, 2001, now abandoned.
Application No. 10/042,527 is a continuation of application No. PCT/GB00/01560, filed on Apr. 20, 2000.
(63) Continuation of application No. PCT/US02/00813, filed on Jan. 11, 2002.
(60) Provisional application No. 60/347,233, filed on Jan. 10, 2002.

(30) Foreign Application Priority Data
Apr. 20, 1999 (GB) 9909066.4
Jan. 12, 2001 (GB) 0100889.5

(51) Int. Cl. A61K 31/445

(52) U.S. Cl. 514/328

(57) ABSTRACT

The use of inhibitors of glucosylceramide synthesis or agents capable of increasing the rate of neuronal glycolipid degradation in the treatment of glycolipid storage related disorders including mucopolysaccharidosis diseases.

### RELATED PATENT APPLICATIONS

This application is a,continuation-in-part of U.S. Ser. No. 10/042,527, filed Oct. 19, 2001, which is, in turn, a continuation of International Application No. PCT/GB00/01560, filed Apr. 20, 2000, which in turn, claims priority from U.K. Application No. 9909066.4, filed Apr. 20, 1999. This is also a continuation of International Application No. PCT/US02/00813, filed Jan. 11, 2002, which claims priority from U.K. Application No. 0100889.5, filed Jan. 12, 2001 and U.S. Provisional Application No. 60/347,233, filed Jan. 10, 2002. All of the above listed applications are herein specifically incorporated by reference in their entireties.

### INTRODUCTION

The present invention relates to the use of inhibitors of glucosylceramide synthesis or agents capable of increasing the rate of neuronal glycolipid degradation in the treatment of glycolipid storage related disorders including mucopolysaccharidosis diseases.

### BACKGROUND OF THE INVENTION

The GM2 gangliosidoses are a group of glycosphingolipid (GSL) lysosomal storage diseases which includes Tay-Sach's disease, Sandhoff's disease and GM2 activator deficiency (Gravel et al (19950 in the Metabolic and Molecular Bases of Inherited Disease (Scriver et al) Vol 2, pp 2839-79, 3 vols, McGraw Hill, New York). They result from mutations in the genes encoding the hexosaminidase a subunit, β subunit and GM2 activator protein respectively. They are characterized by progressive neurodegeneration in response to high levels of lysosomal storage of GM2 and related GSLs, in neurones of the central nervous system (CNS) (Gravel et al (1995) supra). There are currently no therapies for these diseases. Potential therapeutic strategies for Tay-Sach's and Sandhoff's disease include enzyme augmentation and substrate deprivation (Radin (1996) Glycoconj. J. 13:153-7; Platt et al, (1998) Biochemical Pharmacology 56:421-30).

Intravenous administration of mannose-terminated glucocerebrosidase (β-D-glycosyl-N-acylsphingosine glucohydrolase, EC 3.2.1.45) is an effective therapy for Gaucher Type I disease, which is a non-neurological GSL storage disease (Grabowski et al (1995) Ann. Intern. Med. 122:33-39; Beutler et al (1991) Blood 78:1183-9). However, as glycoprotein enzymes fail to cross the blood-brain barrier, this is not a suitable approach for disease involving GSL storage in the CNS. Bone marrow transplantation has the potential to increase enzyme levels in the periphery, and to a limited extent in the CNS due to secretion of enzyme from cells of bone marrow origin, including microglia (Krivit et al (1995) Cell-Transplant 4:385-392). Results of bone marrow transplantation in GSL lysosomal storage diseases involving storage in the CNS have been mixed (Hoogerbrugge et al (1995) Lancet 345:1398-1402). Partial success was recently reported in a mouse model of Sandhoff disease given syngeneic wild type bone marrow (Norfus et al (1998) J. Clin. Invest. 101:1881-8). This led to increased survival of the mice and improved neurological function. Gene therapy also has promise for treating these diseases, although this is currently experimental (Salvetti et al (1995) Br. Med. Bull 51: 106-122). Substrate deprivation is a potentially generic pharmacological approach for treating the GSL storage diseases (Platt et al (1998) Biochemical Pharmacology 56: 421-30), including the G_{M2} gangliosidoses. This strategy is based upon partial inhibition of the ceramide specific glucosyltransferase (glucosylceramide synthase, UDP-glucose:N-acylsphingosine D-glucosyltransferase, EC 2.4.1.80) which catalyses the first step in GSL biosynthesis (Sandhoff et al (1998) Adv. Lipid Res. 26:119-142). This would reduce the levels of GSLs synthesised so they could be catabolised fully by the residual enzyme activity present in the cells.

Substrate deprivation, utilising the GSL biosynthesis inhibitor N-butyldeoxynojirimycin (NB-DNJ), has previously been tested in an in vitro model of Gaucher disease and shown to prevent storage (Platt et al (1994) J. Biol. Chem. 269:8362-6). NB-DNJ has also been evaluated in an asymptomatic mouse model of Tay-Sachs disease and shown to reduce G_{M2} accumulation in the brain and prevent the neuropathology associated with its storage (Platt et al (1997) Science 276:428-31). The imino sugar N-butyldeoxynojirimycin (NB-DNJ) is a potent inhibitor of ceramide glucosyltransferase. NB-DNJ is currently undergoing clinical trials as a treatment for Gaucher and Fabry diseases, glycolipid storage disorders resulting from mutations in glucocerebrosidase and alpha-galactosidase A, respectively.

The galactose analogue of NB-DNJ, N-butyldeoxygalactonojirimycin (NB-DGJ), is known to inhibit GSL synthesis in vitro as effectively as NB-DNJ, but is more specific in that it does not inhibit α-glucosidase I and II or β-glucocerebrosidase (Platt et al, (1994) J Biol Chem 269(43): 27108-14).

Enzyme augmentation could be achieved clinically through strategies such as enzyme replacement, bone marrow transplantation, or gene therapy.

Defects in ganglioside biosynthesis are found in most human cancers and are thought to underlie the invasive and malignant properties of brain tumors (Hakomori (1996) Cancer Res. 56:5309-5318, Fredman et al (1996) Glycoconj. J. 13:391-399).

Glycolipid metabolism also plays a critical role in other neuronal disorders, such as Alzheimer's disease and epilepsy. Niemann-Pick C patient neurons present with fibrillar tangles reminiscent of the morphology seen in Alzheimer's disease. Interestingly, GM1 ganglioside binding by amyloid beta-protein induces conformational changes that support its formation of fibrous polymers, and the fibrillar deposition of this protein is an early event in Alzheimer's disease (Yanigasawa et al (1995) Nat Med 1:1062-6, Choo-Smith et al (1997) Biol. Chem. 272:22987-90). Thus decreasing GM1 synthesis could inhibit the fibre formation seen in Alzheimer's disease.

Disorders which result from accumulation/storage of glucosylceramide containing glycolipids include Gaucher disease, Sandhoff's disease, Fabry's disease, Tay-Sach's disease, Niemann-Pick C storage disease, GM1 gangliosidosis, genetic disorders in which neuronal glycolipid accumulation contributes to the disease pathology, e.g. mucopolysaccharidoses, neurological disorders in which glucosylceramide containing glycolipid accumulation contributes to disease pathology such as Alzheimer's disease, stroke and epilepsy, cancers of neuronal origin such as glioblastoma and astrocytoma and cancers originating outside neuronal tissue but presenting with neuronal metastases.

Mucopolysaccharidosis (MPS) diseases are autosomal recessive disorders of proteoglycan metabolism. A list of the major examples of the currently known mucopolysaccharidosis diseases and the enzyme defects that underlie them is provided in Table 1. The failure to break down proteoglycans may result in the storage of fragments of these materials (glycosaminoglycans (GAGs)) in cells and tissues such as the joints, skeleton, bone marrow, arteries, eyes, skin, lungs, liver, spleen, blood cells and the central nervous system. It should be noted that the extent and location of GAG accumulation occurring in MPS disease depends on the degree to which normal enzyme function is disrupted, and often is somewhat unpredictable.

TABLE 1
Mucopolysaccharidosis diseases

| Common Disease | Disease | | OMIM |
|---|---|---|---|
| Name | Designation | Enzyme Defect | Number* |
| Hurler Syndrome | MPS IH | alpha-L-Iduronidase | 252800 |
| Scheie Syndrome | MPS IS | alpha-L-Iduronidase | 252800 |
| Hurler-Scheie | MPS IH/S | alpha-L-Iduronidase | 252800 |
| Hunter Syndrome | MPS II | Iduronate Sulfatase | 309900 |
| Sanfilippo A | MPS IIIA | Heparan N-Sulfatase | 252900 |
| Sanfilippo B | MPS IIIB | Alpha-N- | 252920 |
| | | Acetylglucosaminidase | |
| Sanfilippo C | MPS IIIC | Acetyl-CoA | 252930 |
| | | Glucosaminide | |
| | | Acetyltransferase | |
| Sanfilippo D | MPS IIID | N-Acetylglucosamine- | 252940 |
| | | 6-Sulfatase | |
| Morquio A | MPS IVA | Galactosamine-6- | 253000 |
| | | Sulfatase | |
| Morquio B | MPS IVB | Beta-Galactosidase | 253010 |
| Maroteaux-Lamy | MPS VI | Arlysulfatase B | 253200 |
| Sly Syndrome | MPS VII | Beta-Glucuronidase | 253220 |

*Online Mendelian Inheritance in Man (www3.ncbi.nlm.nih.gov/omim).

In addition to GAG storage, other materials may accumulate in MPS-affected tissues as well. For example, glycolipid accumulation has been reported in central nervous tissue of man and animals with a variety of MPS diseases (see for example Chapter 136 “The Mucopolysaccharidoses” in The Metabolic and Molecular Bases of Inherited Disease, 8^{th} edition, McGraw-Hill Inc, New York, pp 3421-3452 (2001)). The precise relationship between the effects of the primary storage material, GAGs, and these other materials on the observed disease pathologies is generally uncertain. In fact, the relationship between the storage of GAGs and the symptoms of MPS disease is not clearly understood. For example, diseased neurons from some animal models of MPS exhibit little apparent storage of GAGs. Instead, neurons in these MPS animal models conspicuously store the glycolipids, ganglioside GM2 and GM3 (J. Neurochem., 1994, 62(5), 1852; Neuroscience, 1995, 68(4), 1027 and “Multiple substrate accumulation in MPS disorders and the efficacy of substrate deprivation as therapy” S. Walkley et al, abstract presented at Strategies for Therapy of MPS and Related Diseases, 16^{th} Annual MPS Conference, University of California at Los Angeles, Jun. 21-24, 2001).

The reason for ganglioside storage in MPS diseases is unknown, although secondary inhibition of ganglioside degradative enzymes has been proposed as a possible mechanism. There is as yet no accepted treatment for any of the MPS diseases. A general perception in the art is that the glycolipid accumulation component of MPS disease is a secondary effect of the proteoglycan metabolism defect component (see for example Chapter 136 “The Mucopolysaccharidoses” in The Metabolic and Molecular Bases of Inherited Disease, 8^{th} edition, McGraw-Hill Inc, New York, pp 3421-3452 (2001)).

The use of inhibitors of glucosylceramide synthesis or agents capable of increasing the rate of neuronal glycolipid degradation in the treatment of the cholesterol storage disease, Niemann-Pick disease type C (NPC) has been disclosed in WO 00/62780. Glucosylceramide synthesis inhibitors have been shown to slow the onset of NPC symptoms of neuronal degeneration and to have an effect on the long-term health of NPC animal models, increasing the longevity of treated animals by more than 20% (Current Biology, 2001, 11:1283-1287).

### SUMMARY OF THE INVENTION

The present invention if based, in part, on the discovery that NB-DNJ administered to mice together with glucocerebrosidase (the major therapy for Gaucher Type I patients) unexpectedly does not compromise the activity of glucocerebrosidase, and further, provides an augmentation of enzyme activity over time due to a protective effect of NB-DNJ on the enzyme. This result is surprising as the efficacy of the enzyme would be expected to be compromised in the presence of NB-DNJ, as NB-DNJ is a weak inhibitor of glucocerebrosidase (IC₅₀=0.52 mM). It has further been discovered that the co-administration of NB-DNJ with bone marrow transplantation to provide enzyme augmentation to increase the rate of neuronal glycolipid degradation provides an unexpected synergistic effect.

Accordingly, in one aspect, the invention provides a method for treating a glycolipid storage-related disorder, comprising administering a therapeutically effective amount of an inhibitor of glycolipid synthesis in combination with an agent capable of increasing the rate of glycolipid degradation. In one embodiment, the inhibitor of glucosylceramide synthesis is an imido sugar. In specific embodiments, the inhibitor of glycolipid synthesis and the agent capable of increasing the rate of glycolipid degradation are given simultaneously, sequentially, or separately.

In one embodiment of the method of the invention, the agent capable of increasing the rate of glycolipid degradation is an enzyme involved in glycolipid degradation. In more specific embodiments, the enzyme is selected from the group consisting of glucocerebrosidase, lysosomal hexoseaminidase, galactosidase, sialidase, and glucosylceramide glucosidase. In another embodiment, the agent capable of increasing the rate of neuronal glycolipid degradation is a molecule which increases the activity of a glycolipid degrading enzyme.

In further embodiments, the agent capable of increasing the rate of neuronal glycolipid degradation is a nucleic acid sequence which encodes a neuronal glycolipid degrading enzyme.

In a second aspect, the invention features a method for treating a glycolipid storage-related disorder, comprising administering a therapeutically effective amount of an inhibitor of glycolipid synthesis in combination with bone marrow transplantation.

It has also been found that neuronal glycolipid storage seen in MPS patients may be reduced by treatment with inhibitors of glucosylceramide synthesis or agents capable of increasing the rate of neuronal glycolipid degradation. Reducing ganglioside synthesis through the administration of a glucosylceramide synthesis inhibitor slows MPS disease progression by reducing the interneuronal glycosphingolipid storage burden, a process that would be aided by the fact that ganglioside degredative enzymes in MPS disease cells are believed to be normally produced and trafficked to the lysosome. GM2 and GM3 gangliosides are major secondary storage materials in MPS IIIA disease, accompanying the accumulation of heparan sulfate secondary to sulfamidase deficiency (see S. Walkley, Philosophical Transactions of the Royal Society, B. Biological Sciences, 358:893-904 (2003)). Cholesterol and autofluorescent materials also accumulate in MPS IIIA cells in addition to glycosphingolipids.

Thus, the present invention also provides a method of treating MPS disease, in a mammal in need thereof, comprising administering to said mammal a therapeutically effective amount of an inhibitor of glucosylceramide synthesis.

The invention also provides the use of an inhibitor of glucosylceramide synthesis in the manufacture of a medicament for the treatment of MPS disease.

The invention also provides an inhibitor of glucosylceramide synthesis for use in the treatment of MPS disease.

The MPS disease to be treated according to the invention may be selected from MPS I (MPS IH, IS or IH/S), MPS II, MPS IIIA, IIIB, IIIC or IIID, MPS IVA or IVB, MPS VI or MPS VII.

The invention also provides the use of an inhibitor of glucosylceramide synthesis in the manufacture of a medicament for reducing neuronal glycolipid storage in MPS disease.

The invention also provides a method reducing neuronal glycolipid storage in MPS disease, in a mammal in need thereof, comprising administering to said mammal an inhibitor of glucosylceramide synthesis.

The invention also provides an inhibitor of glucosylceramide synthesis for use in the reduction of neuronal glycolipid storage in MPS disease. More specifically, inhibitors of glucosylceramide synthesis may be used to reduce neuronal glycolipid storage in the brain in MPS disease.

In one embodiment, the inhibitor of glucosylceramide synthesis is an inhibitor of ceramide glucosyltransferase. Inhibitors of glucosylceramide synthesis include small molecules and peptides. Small molecules which may be used in the method of the invention include N-butyldeoxynojirimycin (NB-DNJ), N-butyldeoxygalactonojirimycin (NB-DGJ), and other imino sugar-structured inhibitors of glucosylceramide synthesis such as those disclosed in WO 02/055498. Other small molecule inhibitors of glycosylceramide synthesis include agents such as 1-phenyl-2-decanoylamino-3-morpholino-1-propanol (PDMP), D-threo-1-phenyl-2-decanoylamino-3-morpholino-1-propanol and structurally related analogues thereof.

The definition of inhibitors of glucosylceramide synthesis is also intended to encompass genetic approaches, based on the introduction of nucleic acid coding for proteins or peptides capable of inhibiting glucosylceramide synthesis or antisense sequences or catalytic RNA capable of interfering with the expression of enzymes responsible for glucosylceramide synthesis (e.g. glucosylceramide synthase).

In further embodiments of the method and corresponding use, of the invention, the inhibitor of glucosylceramide synthesis is an antibody capable of binding to and interfering with an enzyme required for glucosylceramide synthesis, for example, glucosylceramide synthase. Thus, the invention extends to the preparation of appropriate constructs and compositions containing antibodies to an enzyme required for glucosylceramide synthesis. In more specific embodiments, the antibodies are polyclonal, monoclonal, or chimeric, including humanized antibodies.

A combination of any of the above inhibitors may be used.

In specific embodiments, compounds for use in the present invention include N-butyldeoxynojirimycin and N-butyldeoxygalactonojirimycin. In a more specific embodiment, the inhibitor compound is N-butyldeoxynojirimycin.

Methods and processes for the production of N-butyldeoxynojirimycin and N-butyldeoxygalactonojirimycin can be found for example in U.S. Pat. No. 4,182,767, EP-B-0012278, EP-A-0624652, U.S. Pat. No. 4,266,025, U.S. Pat. No. 4,405,714 and U.S. Pat. No. 5,151,519.

The invention also provides the use of an agent capable of increasing the rate of neuronal glycolipid degradation in the manufacture of a medicament for the treatment of MPS disease.

The invention also provides a method for the treatment of MPS disease which comprises administering to a subject in need thereof a therapeutically effective amount of an agent capable of increasing the rate of degradation of neuronal glycolipids.

The invention also provides an agent capable of increasing the rate of degradation of neuronal glycolipids for use in the treatment of MPS disease.

Agents capable of increasing the rate of neuronal glycolipid degradation may be used, in particular, to reduce neuronal glycolipid storage in the brain in MPS disease.

Examples of agents capable of increasing the rate of neuronal glycolipid degradation include a nucleic acid sequence (DNA or RNA) which codes for the enzymes which degrade neuronal glycolipids, e.g. glucocerebrosidase, lysosomal hexoseaminidases, galactosidases, sialidases and glucosylceramide glucosidase, i.e. such sequences could be introduced to increase natural production of such enzymes.

In the treatment of MPS, inhibitors of glucosylceramide synthesis or agent capable of increasing the rate of neuronal glycolipid degradation may be co-administered with enzyme replacement therapy, the enzyme replacement therapy being replacement of enzymes known to be defective in MPS diseases, these are listed in Table 1. Specific enzymes which may be mentioned for use in combination therapy according to the invention include alpha-L-iduronidase and iduronate sulfatase.

### BRIEF DESCRIPTION OF THE FIGURES

FIG. 1 is a graph plotting % survival against age of Sandhoff mice in days when treated with different agents.

FIG. 2 shows photomicrographs of brain tissue from A untreated controls (i: upper cortex; ii: lower cortex) and B NB-DNJ treated mice (i: upper cortex; ii: lower cortex).

FIG. 3 shows photomicrographs of the hippocampus from A untreated control or B NB-DNJ treated mice.

FIG. 4 shows photomicrographs of the cerebral cortex from A untreated control or B NB-DNJ treated mice.

FIG. 5 shows photomicrographs of the cerebral cortex from A untreated control or B NB-DNJ treated mice.

### DETAILED DESCRIPTION

Before the present methods, corresponding uses, and compositions are described, it is to be understood that this invention is not limited to particular methods, uses, compositions, and experimental conditions described, as such methods, uses, compositions and corresponding compounds, may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “an inhibitor of glucosylceramide synthesis” includes mixtures of such inhibitors, reference to “the formulation” or “the method” includes one or more formulations, methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

Definitions

In the context of the present invention, the term “inhibitor” of glucosylceramide synthesis includes molecules such as N-butyldeoxynojirimycin or N-butyldeoxygalactonojirimycin, and other imino sugar-structured inhibitors of glucosylceramide synthesis. Also included are agents such as 1-phenyl-2-decanoylamino-3-morpholino-1-propanol (PDMP), D-threo-1-phenyl-2-decanoylamino-3-morpholino-1-propanol and structurally related analogues thereof which exhibit the ability to inhibit glucosylceramide synthesis.

Furthermore, inhibition can also be achieved by the use of genetic approaches, based on the introduction of nucleic acid coding for proteins or peptides capable of inhibiting glucosylceramide synthesis or antisense sequences or catalytic RNA capable of interfering with the expression of enzymes responsible for glucosylceramide synthesis (e.g. glucosylceramide synthase). A combination of any of the above approaches can be used.

The term “substantially pure” when referring to a polypeptide, means a polypeptide that is at least 60%, by weight, free from the proteins and naturally-occurring organic molecules with which it is naturally associated. A substantially pure glucosylceramide synthesis inhibitor is at least 75%, more preferably at least 90%, and most preferably at least 99%, by weight, glucosylceramide synthesis inhibitor. A substantially pure glucosylceramide synthesis inhibitor such as N-butyldeoxynojirimycin (NB-DNJ), can be obtained, for example, by chemical synthesis. Purity can be measured by any appropriate method, e.g. column chromatography, polyacrylamide gel electrophoresis, or HPLC analysis.

“Treatment” refers to the administration of medicine or the performance of medical procedures with respect to a patient, for either prophylaxis (prevention) or to cure the infirmity or malady in the instance where the patient is afflicted.

A “therapeutically effective amount” is an amount of a reagent sufficient to achieve the desired treatment effect.

Pharmaceutical Formulation and Administration

In order to use inhibitors of glucosylceramide synthesis and agents capable of increasing the rate of neuronal glycolipid degradation in therapy, they will normally be formulated into pharmaceutical compositions in accordance with standard pharmaceutical practice.

The medicaments described herein and which are also for use in the methods provided herein, may include one or more of the following: preserving agents, solubilizing agents, stabilizing agents, wetting agents, emulsifiers, sweeteners, colorants, odorants, salts, buffers, coating agents or antioxidants. They may also contain therapeutically active agents in addition to the compounds and/or agents described herein.

The medicaments may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such a composition may be prepared by any method known in the art of pharmacy, for example by admixing the active ingredient with a carrier under sterile conditions.

Small molecule inhibitors of glycosylceramide synthesis are preferably formulated for oral administration.

Oral Administration

Medicaments adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, maize starch or derivatives thereof, stearic acid or salts thereof. Soft gelatine capsules may comprise vegetable oils, waxes, fats, semi-solid, or liquid polyols etc. Solutions and syrups may comprise water, polyols and sugars. For the preparation of suspensions oils (e.g. vegetable oils) may be used to provide oil-in-water or water-in-oil suspensions.

Transdermal Administration

Medicaments adapted for transdermal administration may be provided as discrete patches intended to remain in intimate contact with the epidermis of the recipient for a prolonged period of time. For example, the active ingredient may be delivered from the patch by iontophoresis (Iontophoresis is described in Pharmaceutical Research, 3(6):318 (1986)).

Topical Administration

Medicaments adapted for topical administration may be provided as ointments, creams, suspensions, lotions, powders, solutions, pastes, gels, sprays, aerosols or oils.

For infections of the eye or other external tissues, for example mouth and skin, a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base.

Medicaments adapted for topical administration to the eye include eye drops. Here the active ingredient can be dissolved or suspended in a suitable carrier, e.g. in an aqueous solvent.

Medicaments adapted for topical administration in the mouth include lozenges, pastilles and mouthwashes.

Rectal Administration

Medicaments adapted for rectal administration may be provided as suppositories or enemas.

Nasal Administration

Medicaments adapted for nasal administration which use solid carriers include a coarse powder (e.g. having a particle size in the range of 20 to 500 microns). This can be administered in the manner in which snuff is taken, i.e. by rapid inhalation through the nose from a container of powder held close to the nose. Compositions adopted for nasal administration which use liquid carriers include nasal sprays or nasal drops. These may comprise aqueous or oil solutions of the active ingredient.

Medicaments adapted for administration by inhalation include fine particle dusts or mists, which may be generated by means of various types of apparatus, e.g. pressurized aerosols, nebulizers or insufflators. Such apparatus can be constructed so as to provide predetermined dosages of the active ingredient.

Vaginal Administration

Medicaments adapted for vaginal administration may be provided as pessaries, tampons, creams, gels, pastes, foams or spray formulations.

Parenteral Administration

Medicaments adapted for parenteral administration include aqueous and non-aqueous sterile injectable solutions or suspensions. These may contain antioxidants, buffers, bacteriostats and solutes which render the compositions substantially isotonic with the blood of an intended recipient. Other components which may be present in such compositions include water, alcohols, polyols, glycerine and vegetable oils, for example. Compositions adapted for parenteral administration may be presented in unit-dose or multi-dose containers, for example sealed ampoules and vials, and may be stored in a freeze-dried (lyophilized) condition requiring only the addition of a sterile liquid carrier, e.g. sterile water for injections, immediately prior to use. Extemporaneous injection solutions and suspensions may be prepared from sterile powders, granules and tablets.

The compositions may contain from 0.1% by weight, preferably from 10-60% by weight, of the active material, depending on the method of administration. The amount of the compound of the invention which will be effective in the treatment of mucopolysaccharidosis diseases can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

Dosages will be readily determinable by routine trials, and will be under the control of the physician or clinician. The guiding principle for determining a suitable dose will be delivery of a suitably efficacious but non-toxic, or acceptably toxic, amount of material. The optimal quantity and spacing of individual dosages will be determined by the nature and extent of the condition being treated, the form, route and site of administration, and the particular mammal being treated. It will also be appreciated that the optimal course of treatment, i.e. the number of doses of the active material given per day for a defined number of days, can be ascertained by those skilled in the art using conventional course of treatment determination tests.

For NB-DNJ or a similar compound, a daily dosage for an adult could be expected to be in the range of from 1 mg to 2 g of active agent, and may be in the range of from 50 to 800 mg, or 150 to 600 mg, or 300 to 600 mg. The dosage may be administered in a single daily dose or alternatively in two, three or more doses during the day.

In one embodiment, a nucleic acid comprising a sequence encoding a peptide or protein inhibitor of glucosylceramide synthesis is administered. In another embodiment, a nucleic acid sequence encoding an agent capable of increasing the rate of neuronal glycolipid degradation, e.g. a glucosylceramide glucosidase, is administered. Any suitable methods for administering a nucleic acid sequence available in the art can be used according to the present invention. Methods for administering and expressing a nucleic acid sequence are generally known in the area of gene therapy. For general reviews of the methods of gene therapy, see Goldspiel et al (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; and Morgan and Anderson (1993) Ann. Rev. Biochem. 62:191-217; May (1993) TIBTECH 11(5): 155-215. Methods commonly known in the art of recombinant DNA technology that can be used in the present invention are described in Ausubel et al (eds.), 1993, Current Protocols in Molecular Biology, John Wiley & Sons, NY; and Kriegler (1990) Gene Transfer and Expression, A Laboratory Manual, Stockton Press, NY.

In a particular aspect, the compound comprises a nucleic acid encoding a peptide or protein inhibitor of glucosylceramide synthesis or encoding an enzyme required for neuronal glycolipid degradation, such nucleic acid being part of an expression vector that expresses the peptide or protein in a suitable host. In particular, such a nucleic acid has a promoter operably linked to the coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc. Natl. Acad. Sci. USA 86:8932-8935; Zijlstra et al (1989) Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g. by constructing it as part of an appropriate nucleic acid expression vector and administering it so that it becomes intracellular, e.g. by infection using a defective or attenuated retroviral or other viral vector (see U.S. Pat. No. 4,980,286); by direct injection of naked DNA; by use of microparticle bombardment (e.g. a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g. Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g. PCT Publications WO 92/06180 dated Apr. 16, 1992 (Wu et al); WO 92/22635 dated Dec. 23, 1992 (Wilson et al); WO 92/20316 dated Nov. 26, 1992 (Findeis et al); WO 93/14188 dated Jul. 22, 1993 (Clarke et al); WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al (1989) Nature 342:435-438).

In a further embodiment, a viral vector that contains a nucleic acid encoding a glycolipid degrading enzyme is used, for example, a retroviral vector can be used (see Miller et al (1993) Meth. Enzymol. 217:581-599). These retroviral vectors have been modified to delete retroviral sequences that are not necessary for packaging of the viral genome and integration into host cell DNA. The nucleic acid encoding the enzyme to be used in gene therapy is cloned into the vector, which facilitates delivery of the gene into a subject. More detail about retroviral vectors can be found in Boesen et al (1994) Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are:, Clowes et al (1994) J. Clin. Invest. 93:644-651; Kiem et al (1994) Blood 83:1467-1473; Salmons and Gunzberg (1993) Human Gene Therapy 4:129-141; and Grossman and Wilson (1993) Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (1993) Current Opinion in Genetics and Development 3:499-503 present a review of adenovirus-based gene therapy. Bout et al (1994) Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al (1991) Science 252:431-434; Rosenfeld et al (1992) Cell 68:143-155; Mastrangeli et al (1993) J. Clin. Invest. 91:225-234; WO94/12649; and Wang, et al (1995) Gene Therapy 2:775-783. Adeno-associated virus (AAV) has also been proposed for use in gene therapy (Walsh et al (1993) Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject. In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, and the like. Numerous techniques are known in the art for the introduction of foreign genes into cells (see, e.g. Loeffler and Behr (1993) Meth. Enzymol. 217:599-618; Cohen et al (1993) Meth. Enzymol. 217:618-644; Cline (1985) Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. The resulting recombinant cells can be delivered to a subject by various methods known in the art. In a preferred embodiment, epithelial cells are injected, e.g. subcutaneously. In another embodiment, recombinant skin cells may be applied as a skin graft onto the subject; recombinant blood cells (e.g, hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g. oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g. as obtained from bone marrow, umbilical cord blood, peripheral blood or fetal liver. In a preferred embodiment, the cell used for gene therapy is autologous to the subject that is treated.

In an embodiment in which recombinant cells are used in gene therapy, a nucleic acid encoding a peptide or protein inhibitor of glucosylceramide synthesis, or an agent capable of increasing the rate of neuronal glycolipid degradation is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. WO 94/08598; Stemple and Anderson (1992) Cell 71:973-985; Rheinwald (1980) Meth. Cell Bio. 21A:229; and Pittelkow and Scott (1986) Mayo Clinic Proc. 61:771).

In another embodiment, the nucleic acid to be introduced for purposes of gene therapy may comprise an inducible promoter operably linked to the coding region, such that expression of the nucleic acid is controllable by controlling the presence or absence of the appropriate inducer of transcription.

Direct injection of a DNA coding for a peptide or protein inhibitor of glucosylceramide synthesis or an agent capable of increasing the rate of neuronal glycolipid degradation may also be performed according to, for example, the techniques described in U.S. Pat. No. 5,589,466. These techniques involve the injection of “naked DNA”, i.e. isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA.

In one embodiment of the invention, a mucopolysaccharidosis disease is treated or prevented by administration of a compound that inhibits the expression of one or more enzymes responsible for glucosylceramide synthesis. Compounds useful for this purpose may include antibodies directed to glucosylceramide synthesis enzymes (and fragments and derivatives containing the binding region thereof), and antisense or ribozyme nucleic acids.

In a further embodiment, the expression of an enzyme involved in neuronal glucosylceramide synthesis is inhibited by use of antisense nucleic acids. The present invention provides the therapeutic or prophylactic use of nucleic acids comprising at least six nucleotides that are antisense to a gene or cDNA encoding an enzyme involved in glucosylceramide synthesis or a portion thereof. As used herein, an “antisense” nucleic acid refers to a nucleic acid capable of hybridizing by virtue of some sequence complementarity to a portion of an RNA (preferably mRNA) encoding an enzyme involved in glucosylceramide synthesis. The antisense nucleic acid may be complementary to a coding and/or noncoding region of an mRNA encoding an enzyme involved in glucosylceramide synthesis. Such antisense nucleic acids have utility as compounds that inhibit expression of an enzyme involved in glucosylceramide synthesis, and can be used in the treatment or prevention of neurological disorder.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising a therapeutically effective amount of an antisense nucleic acid that inhibits the expression of an enzyme involved in glucosylceramide synthesis, and a pharmaceutically-acceptable carrier, vehicle or diluent. The antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. The oligonucleotide can be modified at the base moiety, sugar moiety, or phosphate backbone. The oligonucleotide may include other appended groups such as peptides; agents that facilitate transport across the cell membrane (see, e.g. Letsinger et al (1989) Proc. Natl. Acad. Sci. USA 86:6553-6556; Lemaitre et al (1987) Proc. Natl. Acad. Sci. 84:648-652; PCT Publication No. WO 88/09810, published Dec. 15, 1988) or blood-brain barrier (see, e.g. PCT Publication No. WO 89/10134, published Apr. 25, 1988); hybridization-triggered cleavage agents (see, e.g. Krol et al (1988) BioTechniques 6:958-976) or intercalating agents (see, e.g. Zon (1988) Pharm. Res. 5:539-549). In a particular aspect of the invention, an antisense oligonucleotide is provided, preferably of single-stranded DNA. The oligonucleotide may be modified at any position on its structure with substituents generally known in the art.

The antisense oligonucleotide may comprise any suitable of the following modified base moieties, e.g, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 3-(3-amino-3-N-2-carboxypropyl) uracil, (acp3)w, 2,6-diaminopurine, and other base analogs.

In another embodiment, the oligonucleotide comprises at least one modified sugar moiety, e.g. one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, or an analog of formacetal.

In yet another embodiment, the oligonucleotide is an α-anomeric oligonucleotide. An α-anomeric oligonucleotide forms specific double-stranded hybrids with complementary RNA in which, contrary to the usual, β-units, the strands run parallel to each other (Gautier et al (1987) Nucl. Acids Res. 15:6625-6641).

The oligonucleotide may be conjugated to another molecule, e.g. a peptide, hybridization triggered cross-linking agent, transport agent, or hybridization-triggered cleavage agent.

Oligonucleotides of the invention may be synthesized by standard methods known in the art, e.g. by use of an automated DNA synthesizer (such as are commercially available from Biosearch, Applied Biosystems, and the like). As examples, phosphorothioate oligonucleotides may be synthesized by the method of Stein et al (1988) Nucl. Acids Res. 16:3209, and methylphosphonate oligonucleotides can be prepared by use of controlled pore glass polymer supports (Sarin et al (1988) Proc. Natl. Acad. Sci. USA 85:7448-7451).

In another embodiment, the antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Examples of such promoters are outlined above.

The antisense nucleic acids of the invention comprise a sequence complementary to at least a portion of an RNA transcript of a gene encoding an enzyme involved in glucosylceramide synthesis, preferably a human gene encoding an enzyme involved in glucosylceramide synthesis, however, absolute complementarity, although preferred, is not required. A sequence “complementary to at least a portion of an RNA,” as referred to herein, means a sequence having sufficient complementarity to be able to hybridize under stringent conditions (e.g. highly stringent conditions comprising hybridization in 7% sodium dodecyl sulfate (SDS), 1 mM EDTA at 65° C. and washing in 0.1×SSC/0.1% SDS at 68° C., or moderately stringent conditions comprising washing in 0.2×SSC/0.1% SDS at 42° C. with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA encoding an enzyme involved in glucosylceramide synthesis it may contain and still form a stable duplex (or triplex, as the case may be). One skilled in the art can ascertain a tolerable degree of mismatch by use of standard procedures to determine the melting point of the hybridized complex.

Pharmaceutical compositions of the invention, comprising an effective amount of an antisense nucleic acid of the invention in a pharmaceutically acceptable carrier, vehicle or diluent can be administered to a subject having neurological disorder. The amount of antisense nucleic acid which will be effective in the treatment of a neurological disorder can be determined by standard clinical techniques.

In a specific embodiment, pharmaceutical compositions comprising one or more antisense nucleic acids to an enzyme involved in glucosylceramide synthesis are administered via liposomes, microparticles, or microcapsules. In various embodiments of the invention, such compositions may be used to achieve sustained release of the antisense nucleic acids.

Inhibitory Ribozyme and Triple Helix Approaches

In another embodiment, symptoms of MPS disease may be ameliorated by decreasing the level of an enzyme involved in glucosylceramide synthesis by using gene sequences encoding the an enzyme involved in glucosylceramide synthesis in conjunction with well-known gene “knock-out,” ribozyme or triple helix methods to decrease gene expression of an enzyme involved in glucosylceramide synthesis. In this approach ribozyme or triple helix molecules are used to modulate the activity, expression or synthesis of the gene encoding the enzyme involved in glucosylceramide synthesis, and thus to ameliorate the symptoms of the disorder. Such molecules may be designed to reduce or inhibit expression of a mutant or non-mutant target gene. Techniques for the production and use of such molecules are well known to those of skill in the art.

Ribozyme molecules designed to catalytically cleave gene mRNA transcripts encoding an enzyme involved in glucosylceramide synthesis can be used to prevent translation of target gene mRNA and, therefore, expression of the gene product. (See, e.g. PCT International Publication WO90/11364, published Oct. 4, 1990; Sarver et al (1990) Science 247:1222-1225).

Ribozymes are enzymatic RNA molecules capable of catalyzing the specific cleavage of RNA. (For a review, see Rossi (1994) Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g. U.S. Pat. No. 5,093,246, which is incorporated herein by reference in its entirety.

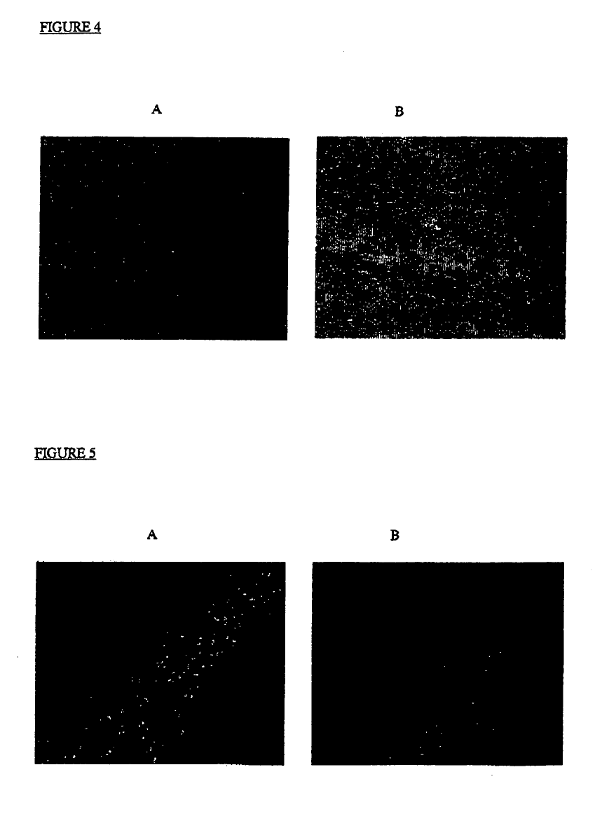

While ribozymes that cleave mRNA at site specific recognition sequences can be used to destroy mRNAs encoding an enzyme involved in glucosylceramide synthesis, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers (1995) Molecular Biology and Biotechnology: A Comprehensive Desk Reference, VCH Publishers, New York, (see especially FIG. 4, page 833) and in Haseloff and Gerlach (1988) Nature, 334, 585-591, each of which is incorporated herein by reference in its entirety.

Preferably the ribozyme is engineered so that the cleavage recognition site is located near the 5′ end of the mRNA encoding the enzyme involved in glucosylceramide synthesis, i.e. to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al (1984) Science, 224, 574-578; Zaug and Cech (1986) Science, 231, 470-475; Zaug, et al (1986) Nature, 324, 429-433; International Ptent Aplication No. WO 88/04300 by University Patents Inc.; Been and Cech (1986) Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the gene encoding the enzyme involved in glucosylceramide synthesis.

As in the antisense approach, the ribozymes can be composed of modified oligonucleotides (e.g. for improved stability, targeting, etc.) and should be delivered to cells that express the enzyme involved in glucosylceramide synthesis in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the enzyme involved in glucosylceramide synthesis and inhibit translation. Because ribozymes, unlike antisense molecules, are catalytic, a lower intracellular concentration is required for efficacy.

Endogenous expression of an enzyme involved in glucosylceramide synthesis can also be reduced by inactivating or “knocking out” the gene encoding an enzyme involved in glucosylceramide synthesis, or the promoter of such a gene, using targeted homologous recombination (e.g. see Smithies et al 1985) Nature 317:230-234; Thomas and Capecchi (1987) Cell 51:503-512; Thompson etal (1989) Cell 5:313-321; and Zijlstra et al (1989) Nature 342:435-438, each of which is incorporated by reference herein in its entirety). For example, a mutant gene encoding a non-functional an enzyme involved in glucosylceramide synthesis (or a completely unrelated DNA sequence) flanked by DNA homologous to the endogenous gene (either the coding regions or regulatory regions of the gene encoding an enzyme involved in glucosylceramide synthesis) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene. However, this approach can be adapted for use in humans provided the recombinant DNA constructs are directly administered or targeted to the required site in vivo using appropriate viral vectors.

Alternatively, the endogenous expression of a gene encoding an enzyme involved in glucosylceramide synthesis can be reduced by targeting deoxyribonucleotide sequences complementary to the regulatory region of the gene (i.e. the gene promoter and/or enhancers) to form triple helical structures that prevent transcription of the gene encoding an enzyme involved in glucosylceramide synthesis in target cells in the body. (See generally, Helene (1991) Anticancer Drug Des. 6(6), 569-584; Helene et al (1992) Ann. N.Y. Acad. Sci., 660, 27-36; and Maher (1992) Bioassays 14(12), 807-815).

Nucleic acid molecules to be used in triplex helix formation for the inhibition of transcription in the present invention should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In one embodiment, wherein the antisense, ribozyme, or triple helix molecules described herein are utilized to inhibit mutant gene expression, it is possible that the technique may so efficiently reduce or inhibit the transcription (triple helix) or translation (antisense, ribozyme) of mRNA produced by normal gene alleles of an enzyme involved in glucosylceramide synthesis that the situation may arise wherein the concentration of such an enzyme involved in glucosylceramide synthesis present may be lower than is necessary for a normal phenotype. In such cases, to ensure that substantially normal levels of activity of a gene encoding an enzyme involved in glucosylceramide synthesis are maintained, gene therapy may be used to introduce into cells nucleic acid molecules that encode and express an enzyme involved in glucosylceramide synthesis that exhibit normal gene activity and that do not contain sequences susceptible to whatever antisense, ribozyme, or triple helix treatments are being utilized. Alternatively, in instances whereby the gene encodes an extracellular protein, a normal enzyme can be co-administered in order to maintain the requisite level of activity.

Antisense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Use of Antibodies as Inhibitors of Glycosylceramide Synthesis

According to one embodiment of the invention, a mucopolysaccharidosis disease is treated or prevented by administration of one or more antibodies that inhibit the expression of one or more enzymes responsible for glucosylceramide synthesis. Any of the enzymes required for glucosylceramide synthesis, or a fragment or derivative thereof, may be used as an immunogen to generate antibodies which immunospecifically bind such an immunogen. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e. molecules that contain an antigen binding site that specifically binds an antigen. The immunoglobulin molecules of the invention can be of any class (e.g. IgG, IgE, IgM, IgD and IgA) or subclass of immunoglobulin molecule.

In one embodiment, antibodies that recognize an enzyme required for glucosylceramide synthesis may be prepared. For example, antibodies may be prepared that recognize glucosylceramide glucosyltransferase, glucosylceramide synthase, etc. Alternatively, such antibodies may be purchased from commercial sources where available. In another embodiment, methods known to those skilled in the art are used to produce antibodies that recognize an enzyme required for glucosylceramide synthesis.

In the production of antibodies, screening for the desired antibody can be accomplished by techniques known in the art, e.g. ELISA (enzyme-linked immunosorbent assay). For example, to select antibodies which recognize a specific domain of an enzyme required for glucosylceramide synthesis, one may assay generated hybridomas for a product which binds to an enzyme fragment containing such domain. Polyclonal antibodies which may be used in the methods of the invention are heterogeneous populations of antibody molecules derived from the sera of immunized animals. Unfractionated immune serum can also be used. Various procedures known in the art may be used for the production of polyclonal antibodies to a protein enzyme required for glucosylceramide synthesis, or a peptide or fragment thereof. In a particular embodiment, rabbit polyclonal antibodies to an epitope of such an immunogen polypeptide can be obtained. For example, for the production of polyclonal or monoclonal antibodies, various host animals can be immunized by injection with the native or a synthetic (e.g. recombinant) version of an enzyme required for glucosylceramide synthesis, or peptide or fragment thereof, including but not limited to rabbits, mice, rats, etc. Isolated enzymes suitable for such immunization may be obtained by the use of discovery techniques, such as the preferred technology described herein. If the enzyme is purified by gel electrophoresis, the enzyme can be used for immnunization with or without prior extraction from the polyacrylamide gel. Various adjuvants may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward an enzyme required for glucosylceramide synthesis, any technique which provides for the production of antibody molecules by continuous cell lines in culture may be used. For example, the hybridoma technique originally developed by Kohler and Milstein (1975) Nature 256:495-497), as well as the trioma technique, the human B-cell hybridoma technique (Kozbor et al (1983) Immunology Today 4:72), and the EBV-hybridoma technique to produce human monoclonal antibodies (Cole et al (1985) in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g. human-mouse chimeras). Humanized antibodies are antibody molecules from non-human species having one or more complementarily determining regions (CDRs) from the non-human species and a framework region from a human immunoglobulin molecule. (See, e.g. U.S. Pat. No. 5,585,089, which is incorporated herein by reference in its entirety.)

Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al (1988) Science 240:1041-1043; Liu et al (1987) Proc. Natl. Acad. Sci. USA 84:3439-3443; Liu et al (1987) J. Immunol. 139:3521-3526; Sun et al (1987) Proc. Natl. Acad. Sci. USA 84:214-218; Nishimura et al 1987) Canc. Res. 47:999-1005; Wood et al (1985) Nature 314:446-449; and Shaw et al (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al 1986) Nature 321:552-525; Verhoeyan et al (1988) Science 239:1534; and Beidler et al (1988) J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with selected antigens, e.g. all or a portion of glucosylceramide synthase. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g. U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g. a mouse antibody, is used to guide the selection of a completely human antibody recognizing the same epitope. (Jespers et al (1994) Biotechnology 12:899-903).

The antibodies of the present invention can also be generated using various phage display methods known in the art. In phage display methods, functional antibody domains are displayed on the surface of phage particles which carry the polynucleotide sequences encoding them. In a particular, such phage can be utilized to display antigen binding domains expressed from a repertoire or combinatorial antibody library (e.g. human or murine). Phage expressing an antigen binding domain that binds the antigen of interest can be selected or identified with antigen, e.g. using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al (1995) J. Immunol. Methods 182:41-50; Ames et al (1995) J. Immunol. Methods 184:177-186; Kettleborough et al (1994) Eur. J. Immunol. 24:952-958; Persic et al (1997) Gene 187 9-18; Burton et al (1994) Advances in Immunology 57:191-280; PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, inciuding human antibodies, or any other desired antigen binding fragment, and expressed in any desired host, including mammalian cells, insect cells, plant cells, yeast, and bacteria, e.g. as described in detail below. For example, techniques to recombinantly produce Fab, Fab′ and F(ab′)2 fragments can also be employed using methods known in the art such as those disclosed in PCT Publication No. WO 92/22324; Mullinax et al (1992) BioTechniques 12(6):864-869; and Sawai et al (1995) AJRI 34:26-34; and Better et al Science (1988) 240:1041-1043 (said references incorporated by reference in their entireties).

Examples of suitable techniques which can be used to produce single-chain Fvs and antibodies against APIs of the present invention include those described in U.S. Pat. Nos. 4,946,778 and 5,258,498; Huston et al (19910 Methods in Enzymology 203:46-88; Shu et al (1993) PNAS 90:7995-7999; and Skerra et al (1988) Science 240:1038-1040.

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al (1983) Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published 13 May 1993, and in Traunecker et al (1991) EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al. (1986) Methods in Enzymology 121:210.

The invention provides functionally active fragments, derivatives or analogs of the specific immunoglobulin molecules. Functionally active means that the fragment, derivative or analog is able to elicit anti-anti-idiotype antibodies (i.e. tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any suitable binding assay known in the art.

The present invention provides antibody fragments such as, but not limited to, F(ab′)2 fragments and Fab fragments. Antibody fragments which recognize specific epitopes may be generated by known techniques. F(ab′)2 fragments consist of the variable region, the light chain constant region and the CH1 domain of the heavy chain and are generated by pepsin digestion of the antibody molecule. Fab fragments are generated by reducing the disulfide bridges of the F(ab′)2 fragments. The invention also provides heavy chain and light chain dimers of the antibodies of the invention, or any minimal fragment thereof such as Fvs or single chain antibodies (SCAs) (e.g. as described in U.S. Pat. No. 4,946,778; Bird (1988) Science 242:423-42; Huston et al (1988) Proc. Natl. Acad. Sci. USA 85:5879-5883; and Ward et al (1989) Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al (1988) Science 242:1038-1041).

In other embodiments, the invention provides fusion proteins of the immunoglobulins of the invention (or functionally active fragments thereof), for example in which the immunoglobulin is fused via a covalent bond (e.g. a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are either modified, i.e. by the covalent attachment of any type of molecule as long as such covalent attachment that does not impair immunospecific binding. For example, but not by way of limitation, the derivatives and analogs of the immunoglobulins include those that have been further modified, e.g. by glycosylation, acetylation, pegylation, phosphylation, amidation, derivatization by known protecting/blocking groups, proteolytic cleavage, linkage to a cellular ligand or other protein, or the like. Any of numerous chemical modifications may be carried out by known techniques, including, but not limited to specific chemical cleavage, acetylation, formylation, etc. Additionally, the analog or derivative may contain one or more non-classical or unnatural amino acids.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Co-Administration of Ceredase™ and NB-DNJ

A group of mice were treated with NB-DNJ at 4800 mg/kg/day for 5 weeks. After a low intravenous dose (5-10 U/kg) of Ceredase™ (Genzyme Corporation) administered as a single injection via the tail vein, serum enzyme activity was measured by taking sequential serum samples from the tail vein to monitor enzyme activity over time. Ceredase™ is a modified form of β glucocerebrosidase. The results are shown in Table 2.

TABLE 2
Effect of NB-DNJ on circulatory activity and half life of Ceredase ™

| Mouse | Peak Activity | T_{1/2} (min) |
|---|---|---|
| Control | | |
| 1 | 5.8 | 4.2 |
| 2 | 7.9 | 3.3 |
| 3 | 8.0 | 1.5 |
| 4 | 6.8 | 1.8 |
| 5 | 30.0 | 1.4 |
| 6 | 2.8 | 2.0 |
| 7 | 13.6 | 1.2 |
| 8 | 17.6 | 1.2 |
| Mean ± sem | 11.6 ± 3.1 | 2.1 ± 0.4 |
| NB-DNJ | | |
| 1 | 13.9 | 1.7 |
| 2 | 32.1 | 4.9 |
| 3 | 24.1 | 5.3 |
| 4 | 13.1 | 3.0 |
| 5 | 21.0 | 3.5 |
| 6 | 68.3 | 2.4 |
| 7 | 19.2 | 2.8 |
| Mean ± sem | 27.4 ± 7.2 | 3.4 ± 0.5 |

Ceredase™ activity and serum half lives appeared to be increased in mice treated with NB-DNJ, suggesting a protective effect of the compound to enzyme clearance. It was concluded that (a) co-administration of NB-DNJ with Ceredase™ does not compromise activity and (b) there is a surprising augementation of enzyme activity over time due to a protective effect of the compound on the enzyme.

### Example 2

### Co-Administration of NB-DNJ and Bone Marrow Transplantation in a Mouse Model of Sandoff Disease

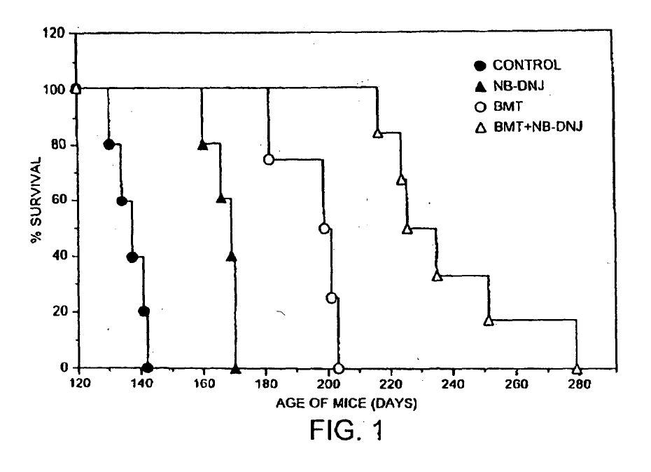

Sandhoff mice were bone marrow transplanted at two weeks of age and drug therapy initiated at 9.5-11 weeks of age (600 mg/kg/day). Survival curves were plotted for each group of animals with each point on the graph representing a death (FIG. 1). The untreated (no BMT, no drug) survived (longest survivor) until 140 days (filled circles), NB-DNJ only (no BMT) survived until 170 days, BMT only (no NB-DNJ) survived until 200 days, and NB-DNJ plus BMT had extended survival from 200-280 days. The data show synergy approximately 13% above additive.

### Example

### Inhibition of Clinical and Pathological Symptoms in a Murine Model of MPS

A murine model of MPS IIIA (Sanfilippo disease) has been described that demonstrates the disorder's characteristic joint and skeletal storage of proteoglycan fragments, and neuronal storage of GM2 and GM3 gangliosides. Colonies of mutant mice expressing the MPS IIIA phenotype have been described, and have been validated by a number of criteria as an authentic model of the disease (Stanley, P et al (1999) Glycobiology 9: 1389-1396). MPS IIIA mice display clinical signs of the disease around 6 months of age with decreased activity, scruffy coat, abdominal distention, hunched posture and waddling gait. By 12 months, the mice exhibit severe ataxia, tremors and weight loss. Death results by 18 months or less.

The brains of MPS mice are grossly normal. However, microscopic examination reveals swollen somata, meganeurite formation and enlarged axon hillock regions of cortical pyramidal neurons. White matter and Purkinje cells display axonal spheroids. Anti-heparan sulfate immunostaining indicates accumulation of heparan sulfate fragments in the cerebral cortex of MPS IIIA animals. Anti-ganglioside antibody staining shows increased GM2 and GM3 levels primarily in laminae II/III and V pyramidal neurons, and in other neurones of the central nervous system.

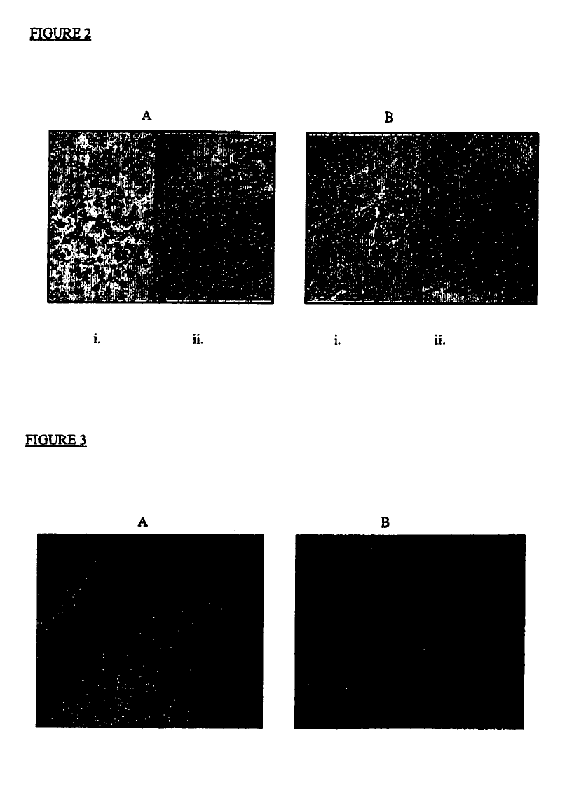

MPS IIIA mice were treated with 1200 mg/kg/day, beginning at 4 weeks of age, with NB-DNJ. Brain samples from animals were taken at 8 weeks of age (4 weeks of treatment with NB-DNJ) and subjected to immunocytochemistry with an anti-GM2 ganglioside-specific monoclonal antibody. FIG. 2 shows photomicrographs of: A: Untreated control, i. Upper cortex, ii. Lower cortex; B: NB-DNJ Treated, i. Upper cortex, ii. Lower cortex.

The hippocampus (CA4-Dentate Gyrus) of the MPS IIIA animals was also examined for staining by a GM2 Ganglioside-specific monoclonal antibody. FIG. 3 shows photomicrographs of: A: Untreated control; B: NB-DNJ Treated.

Cerebral cortex samples from animals were also taken at 40 weeks of age (36 weeks of treatment with NB-DNJ) and subjected to immunocytochemistry with an anti-GM3 ganglioside-specific monoclonal antibody. Anti-GM2 staining was similar to anti-GM3 staining in both treated and untreated animals, respectively (data not shown). FIG. 4 shows photomicrographs of the anti-GM3 staining of: A: Untreated control; B: NB-DNJ Treated.

The brains of animals affected by MPS IIIA, as well as some other of the mucopolysaccharidoses, store heterogeneous auto-fluorescent material in addition to glycolipids. Thus, the cerebral cortex of MPS IIIA animals either left untreated or treated for 36 weeks with NB-DNJ were also examined for the presence of auto-fluorescent material. It was found that NB-DNJ reduced the storage of auto-fluorescent material in MPS IIIA animals. FIG. 5 shows photomicrographs of: A: Untreated control; B: NB-DNJ Treated.

Conclusions: These data demonstrate that the glycolipid storage occurring in the neuronal cells of an animal model of MPS IIIA is markedly reduced by treatment with a glycolipid synthesis inhibitor. NB-DNJ markedly reduces the pathological features in the model of MPS IIIA.

Thus, these data demonstrate that glycolipid synthesis inhibitors provide a useful treatment option for the pathologies resulting from glycolipid accumulation in MPS-affected brains.

1-20. (Canceled)

21. A method of treating mucopolysaccharide disease in a patient in need thereof comprising administering a therapeutically effective amount of an inhibitor of glucosylceramide synthesis.

22. The method according to claim 21 wherein the mucopolysaccharide disease is selected from the group consisting of MPS I (MPS IH, IS or IH/S), MPS II, MPS IIIA, IIIB, IIIC or IIID, MPS IVA or IVB, MPS VI and MPS VII.

23. The method according to claim 21 wherein the inhibitor is an inhibitor of ceramide glucosyltransferase.

24. The method according to claim 21 wherein the inhibitor is an imino sugar.

25. The method according to claim 24 wherein the inhibitor is N-butyldeoxynojirimycin or N-butyldeoxygalactonojirimycin.

26. The method according to claim 25 wherein the inhibitor is N-butyldeoxynojirimycin.

27. The method according to claim 21 wherein the inhibitor is a nucleic acid coding for a protein or peptide capable of inhibiting glucosylcerarnide synthesis, or an antisense sequence or catalytic RNA capable of interfering with the expression of enzymes responsible for glucosylceramide synthesis.

28. A method of reducing neuronal glycolipid storage in mucopolysaccharide disease in a patient in need thereof comprising administering a therapeutically effective amount of an inhibitor of glucosylceramide synthesis.

29. The method according to claim 28 wherein the mucopolysaccharide disease is selected from the group consisting of MPS I (MPS IH, IS or IH/S), MPS II, MPS IIIA, IIIB, IIIC or IIID, MPS IVA or IVB, MPS VI and MPS VII.

30. The method according to claim 28 wherein the inhibitor is an inhibitor of ceramide glucosyltransferase.

31. The method according to claim 28 wherein the inhibitor is an imino sugar.

32. The method according to claim 31 wherein the inhibitor is N-butyldeoxynojirimycin or N-butyldeoxygalactonojirimycin.

33. The method according to claim 32 wherein the inhibitor is N-butyldeoxynojirimycin.

34. The method according to claim 28 wherein the inhibitor is a nucleic acid coding for a protein or peptide capable of inhibiting glucosylceramide synthesis, or an antisense sequence or catalytic RNA capable of interfering with the expression of enzymes responsible for glucosylceramide synthesis.

35. A method of treating mucopolysaccharide disease in a patient in need thereof comprising administering a therapeutically effective amount of an agent capable of increasing the rate of neuronal glycolipid degradation.
